(19) United States
(12) Patent Application Publication
(10) Pub. No.: US 2024/0150719 A1
(43) Pub. Date: May 9, 2024

(54) MAGNETIC SHEAR BIOREACTOR APPARATUS AND METHODS

(71) Applicant: THE BOARD OF REGENTS OF THE UNIVERSITY OF TEXAS SYSTEM, Austin, TX (US)

(72) Inventors: Brijesh S. GILL, Houston, TX (US); Charles S. COX, Jr., Houston, TX (US)

(21) Appl. No.: 18/547,408

(22) PCT Filed: Feb. 21, 2022
(86) PCT No.: PCT/US2022/017164

§ 371 Date: Aug. 22, 2023

Related U.S. Application Data
(60) Provisional application No. 63/151,968, filed on Feb. 22, 2021.

(51) Int. Cl. C12N 5/0775; C12M 1/42; C12M 3/00; C12M 3/04; C12N 5/00; C12N 13/00

(52) U.S. Cl. CPC C12N 5/0662; C12M 23/48; C12M 27/10; C12M 35/04; C12M 35/06; C12N 5/0075; C12N 13/00; C12N 2527/00

(57) ABSTRACT

Apparatus and methods for culturing cells in a cell culture medium including magnetic beads in a fluid. A variable magnetic field can be applied to the magnetic beads to create shear forces on cells on the surface of the beads. In certain embodiments, a rotational force can also be applied to the magnetic beads and the magnetic force can counteract the rotational force.

### CROSS-REFERENCE TO RELATED APPLICATIONS

This application claims the benefit of U.S. Provisional Patent Application No. 63/151,968, filed Feb. 22, 2021, the entirety of which is incorporated herein by reference.

### BACKGROUND OF THE INVENTION

### Field of the Invention

Embodiments of the present invention relate to methods and apparatus for conditioning cell populations for improved characteristics for use as therapeutic agents. More specifically, the embodiments of the present invention relate to an apparatus and a method for conditioning stem cells by applying magnetic and/or rotational forces to impose a controlled shear stress on stem cells disposed along a boundary of a flow chamber in which the stem cells are disposed.

### Background of the Related Art

Lineage specific differentiated cell populations are contemplated for use in cell replacement therapies for patients with diseases or disorders. Cell populations that retain the ability to differentiate into specialized cell types (stem cells) and/or secrete certain factors have been contemplated for use in cell-based therapies for patients with a variety of diseases or disorders.

There is a need for the ability to obtain sufficient donor cell populations that are reliably conditioned such that they are predictable in their therapeutic activity. The present methods and apparatus provide a solution to these problems and thus facilitate the use of cells as cellular therapeutics or as the source of soluble factors.

A bioreactor is a device in which components of biological materials, such as stem cell-containing fluids, may be conditioned by manipulation of the factors that influence the materials. The condition of a stem cell-containing fluid is influenced by multiple factors including pH, waste content, nutrient content and the type and concentration of dissolved gases such as, for example, oxygen. These factors may generally be referred to as chemical factors that influence the condition of stem cells in a stem cell-containing fluid.

A bioreactor may enable the manipulation of the condition of the stem cell-containing fluids by control of non-chemical factors. Conventional apparatuses and methods of conditioning cell populations for conventional cell therapies fail to enable the precise control of mechanical shear to the cells to be conditioned. Apparatus and methods for controllably applying shear stress to cells to be conditioned within a bioreactor flow chamber capable of large-scale cell production are therefore desired.

### SUMMARY OF THE INVENTION

Embodiments of the present disclosure include apparatus and methods to address shortcomings in existing systems. Specific embodiments can apply magnetic and/or rotational fluid forces to apply shear stress to cells in a bioreactor.

Certain embodiments include an apparatus comprising a stationary member, a magnetic field generator, a controller and a cell culture medium located in the stationary member, wherein the cell culture medium comprises magnetic beads in a fluid, and the controller is configured to control the magnetic generator to generate a variable magnetic field on the magnetic beads. Particular embodiments further comprise a rotating member, wherein the rotating member is configured to rotate within the stationary member. In some embodiments, the cell culture medium is located between the stationary member and the rotating member. In specific embodiments the rotating member applies a rotational force to the magnetic beads via the fluid of the cell culture medium, wherein the rotational force is in a first direction, and the variable magnetic field applies a magnetic force to the magnetic beads, wherein the magnetic force is in a second direction that is different than the first direction.

In certain embodiments the first direction is perpendicular to the second direction, and in particular embodiments the magnetic force applied to the magnetic beads is greater than the rotational force applied to the magnetic beads. In some embodiments the variable magnetic field is a pulsed magnetic field. In specific embodiments the rotating member comprises a plurality of discs. In certain embodiments the stationary member comprises a plurality of annular surfaces, and in particular embodiments the plurality of annular surfaces are interdigitated with the plurality of discs. Some embodiments furthering comprise apertures extending through the plurality of discs. In specific embodiments the variable magnetic field moves the magnetic beads through the apertures extending through the plurality of discs.

In certain embodiments the rotating member comprises a plurality of randomly oriented fibers and in particular embodiments the stationary member is configured as a toroidal container or a linear tubular container. In some embodiments the magnetic field generator is configured as a series of coils wrapped around the toroidal container or the linear tubular container. In specific embodiments the controller is configured to pulse an electrical current through the series of coils wrapped around the toroidal container or the linear tubular container. In certain embodiments the magnetic beads are moved around the toroidal container or within the linear tubular container via the electrical current pulsed through the series of coils.

Particular embodiments include a method of culturing cells, where the method comprises: obtaining a cell culture medium comprising magnetic beads in a fluid, and applying a variable magnetic force to the magnetic beads. In some embodiments the variable magnetic force is a pulsed magnetic force. Specific embodiments further comprise applying a rotational force to the magnetic beads via the fluid of the cell culture medium.

In particular embodiments the rotational force is applied to the magnetic beads in a first direction, and the variable magnetic force is applied to the magnetic beads in a second direction that is different than the first direction. In some embodiments the first direction is perpendicular to the second direction. In specific embodiments the magnetic force applied to the magnetic beads is greater than the rotational force applied to the magnetic beads. In certain embodiments the rotational force is applied by a rotating member comprising a plurality of discs.

In particular embodiments the cell culture medium is contained in a stationary member comprising a plurality of annular surfaces. In some embodiments the plurality of annular surfaces are interdigitated with the plurality of discs. In specific embodiments the plurality of discs comprises apertures extending through the plurality of discs; and the variable magnetic field moves the magnetic beads through the apertures extending through the plurality of discs.

Some embodiments further comprise rotating a rotating member comprising a plurality of randomly oriented fibers to apply a rotational force to the magnetic beads via the fluid of the cell culture medium. In specific embodiments the cell culture medium comprising magnetic beads in the fluid is contained in a toroidal container. In certain embodiments the variable magnetic force is applied to the magnetic beads via a magnetic field generator configured as a series of coils wrapped around the toroidal container. Specific embodiments further comprise pulsing an electrical current through the series of coils wrapped around the toroidal container. Certain embodiments further comprise moving the magnetic beads around the toroidal container via the electrical current pulsed through the series of coils.

Further aspects of the embodiments disclose the application of shear stress in a bioreactor (e.g., such as a bioreactor apparatus as detailed herein).

Certain embodiments of the invention concern a method of producing a conditioned composition. As used herein, a conditioned composition refers to a composition that has been subjected to the conditioning effects of mechanical forces. For example, the mechanical force can be an application of controlled shear stress with a force sufficient to produce a conditioned composition. In some aspects, the conditioned composition comprises a population of conditioned pluripotent cells (e.g., MSCs). Thus, certain aspects concern the isolation of a population of conditioned pluripotent cells. In further aspects, the conditioned composition is a media (e.g. a cell-free media) comprising secreted factors from pluripotent cells that have been subjected to a controlled sheer stress.

Aspects of the embodiments involve culturing of the stem cells on a substrate to allow cell adhesion. In some cases, the substrate is a surface that supports the growth of the stem cells in a monolayer. For example, in some aspects, the surface is a plastic or glass surface, such as a surface that has been coated with extracellular matrix materials (e.g., collagen IV, fibronectin, laminin and/or vitronectin). In further aspects, the substrate may be modified to incorporate a surface (or surface coating) with increased or decreased surface energy. Examples of low energy materials that may be used as a surface or surface coating include, without limitation, hydrocarbon polymers, such as polyethylene, or polypropylene and nitrides. For instance, a surface or surface coat may comprise Polyhexafluoropropylene, Polytetrafluoroetylene, Poly(vinylidene fluoride), Poly(chlorotrifluoroethylene), Polyethylene, Polypropylene, Poly(methylmethacrylate)—PMMA, Polystyrene, Polyamide, Nylon-6,6, Poly(vinylchloride), Poly(vinylidene chloride), Poly(ethylene terephthalate), Epoxy (e.g., rubber toughened or amine-cured), Phenol-resorcinol resin, Urea-formaldehyde resin, Styrene-butadiene rubber, Acrylonitrile-butadiene rubber and/or Carbon fiber reinforced plastic. Examples of high energy materials that may be used as a surface or surface coating include, without limitation, metals and oxides. For instance, a surface or surface coat may comprise Aluminum oxide, Beryllium oxide, Copper, Graphite, Iron oxide (Fe2O3), Lead, Mercury, Mica, Nickel, Platinum, Silicon dioxide—silica and/or Silver.

Further aspects of the embodiments concern the application of controlled shear stress with a force sufficient to produce a conditioned composition. In certain aspects, the shear stress is applied in the form of fluid laminar shear stress. For example, the force of the shear stress is at least about 1, 2, 3, 4, 5, 6, 7, 8, 9 or 10 dynes per square centimeter. In certain instances, the force of the shear stress is at least 5, 10 or 15 dynes per square centimeter, such as between about 5-20; 5-15; 10-20 or 10-15 dynes per square centimeter. In some aspects, the controlled shear stress is applies for a period of between about 1 minute and two days. For example, the controlled shear stress can be applied for a period of between 5 minutes to 24 hours; 10 minutes to 24 hours; 0.5 hours to 24 hours or 1 hour to 8 hours. In further aspects, cells of the embodiments are exposed to an elevated pressure.

In still further aspects, cells or media from a culture of the embodiments are tested periodically to determine the level of conditioning. For example, a sample comprising cells or media can be taken from the culture about every 10 minutes, 15 minutes, 30 minutes or every hour. Such samples may be tested, for example to determine the expression level of anti-inflammatory factors, such as transcription factors or cytokines. In particular aspects, cells or media can be taken from a sampling port positioned in a location of lower fluid pressure, including for example near the inlet of a pump configured to direct fluid through the apparatus.

In certain aspects, a starting population of stem cells is obtained. For example, the starting stem cell population can comprise induced pluripotent stem (iPS) cells or mesenchymal stem cells (MSCs). In some aspects, the MSCs are isolated from tissue. For example, in some aspects, the tissue comprises bone marrow, cord blood, peripheral blood, fallopian tube, fetal liver, lung, dental pulp, placenta, adipose tissue, or amniotic fluid. In further aspects the cells are human cells. For example, the cells can be autologous stem cells. In some aspects, the stem cells are transgenic cells.

In further aspects of the embodiments, a method of producing a conditioned composition comprises passing fluid over the stem cells for the application of controlled shear stress. For example, the fluid passed over the stem cells can be a cellular growth medium. In some aspects, the growth medium comprises at least a first exogenous cytokine, growth factor, TLR agonist or stimulator of inflammation. For example, the growth medium can comprise IL1B, TNF-α, IFNγ, PolyI:C, lipopolysaccharide (LPS), phorbol myristate acetate (PMA) and/or a prostaglandin. In certain specific aspects, the prostaglandin is 16,16′-dimethyl prostaglandin E2 (dmPGE2).

In certain aspects, conditioned stem cells of the embodiments have at least 2-, 3-. 4-. 5- or 6-fold higher expression of an anti-inflammatory gene compared to the starting populating of stem cells. For example, the anti-inflammatory gene can be TSG-6, PGE-2, COX-2, IL1Ra, HMOX-1, LIF, and/or KLF2.

In further embodiments, the conditioned composition comprises a conditioned media composition. Thus, certain aspects concern the isolation of conditioned media after the application of shear stress. In some cases, the conditioned media is essentially free of cells.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The objects and advantages of the present methods, system and apparatus will be better understood and more readily apparent when considered in conjunction with the following detailed description and accompanying drawings which illustrate, by way of example, preferred embodiments of this system and methods.

FIG. 1 is an exploded schematic view of a bioreactor apparatus according to the present disclosure.

FIG. 2 is a partial section view of the embodiment of FIG. 1.

FIG. 3 is a first perspective view a rotating member of the embodiment of FIG. 1.

FIG. 4 is a second perspective view the rotating member of FIG. 3.

FIG. 5 is a perspective view of a portion of a stationary member according to the present disclosure.

FIG. 6 is section view of the rotating member of FIG. 3-4 and the stationary member of FIG. 5.

FIG. 7 is a perspective view a rotating member according to the present disclosure.

FIG. 8 is a perspective view of an apparatus according to the present disclosure.

FIG. 9 is a partial section view of an apparatus according to the present disclosure.

FIG. 10 graphs demonstrate that transcriptional induction is robust for COX2, TSG6, HMOX1, and IL1RN in fluid shear stressed human MSCs. hBM MSC, human bone marrow MSC; hAF MSC, amniotic fluid MSC; hAD MSC, adipose-derived MSC. P-values calculated by paired t-test, equal variance.

FIG. 11 illustrates representative Western blots showing increased intracellular COX2 protein, which is reduced by NF-kB antagonist BAY11-7085 (10 μM).

FIG. 12 illustrates TNF-αcytokine suppression assays that highlight functional enhancement of MSC immunomodulation. Human MSCs preconditioned by mechanical force (15 dyne/cm2 shear stress for 3 hours) are placed in static co-culture with lipopoly-saccharide (LPS) or phytohaemagglutinin (PHA)-activated splenocytes including macrophages, neutrophils, NK, B, and T cells. Results of the assays show that TNF-α secretion by splenocytes is reduced by 10-50% when MSCs are transiently conditioned with shear stress. Lower values correspond to greater anti-inflammatory potency. hBM MSC, human bone marrow MSC; hAF MSC, amniotic fluid MSC; hAD MSC, adipose-derived MSC. P-values calculated by paired t-test, equal variance.

FIG. 13 illustrates inhibition of COX2 (Indomethacin, 10 μM; NS-398, 10 μM) and NF-kB (BAY11-7085, 10 μM) abrogate the positive effects of shear stress, whereas ectopic dmPGE2 (10 μM) mimics MSC suppression. Asterisks indicate p<0.001 compared to Static Vehicle. n=6 indicates six different MSC donor lines included in data shown.

FIG. 14 illustrates that priming agents complement shear-induced anti-inflammatory signaling. Darkened bars represent treatment combinations that indicate a substantial induction of the pathway by shear stress and cytokines (IFN-γ, 20 ng/ml; TNF-α, 50 ng/ml). In this low-performance human amniotic MSC line, TSG6 was induced by shear stress only. IDO was induced by IFN-γ only. hAF MSC, human amniotic fluid MSC.

### DESCRIPTION OF EXEMPLARY EMBODIMENTS

When dealing with multipotent stem cells, such as mesenchymal stem cells (MSCs), generating consistent results requires precise control of the cell's environment, which includes a number of parameters. These include chemical factors such as nutrients, waste products, pH and dissolved gases. Many state of the art bioreactors have been designed and built to control and monitor these chemical parameters.

However, the mechanical environment also plays a significant role in the outcome of a stem cell. The mechanical environment is dependent upon two major elements: the substrate and the dynamic state of the surrounding media. Substrate stiffness can be controlled through careful selection of surface treatments or coatings, which can influence the adherence of the cells to the substrate.

After a population of cells has adhered to a substrate, they are subjected to mechanical forces applied by the media in which the cells are immersed. In conventional cell culture, these forces are essentially zero since there is no bulk flow of media. Existing bioreactor systems have constant or variable fluid flow (media), but lack design control of the pattern of flow (laminar-uni or bi-directional vs. turbulent) or the degree of shear forces that interact with the cells. Moving fluid exerts on cells a shear stress that is proportional to the fluid velocity and viscosity. Most mechanotransduction studies have been performed in the laminar flow regime where shear stress behavior is well known and characterized by simple equations. Laminar flow is characterized by a non-uniform velocity profile across the cross-section of the flow channel. Fluid velocity at the boundaries (e.g. walls) can be assumed to be zero, known as the “no-slip condition” or “boundary condition.” A straightforward equation describing shear stress ( [symbol] ) for Newtonian fluids in laminar flow is [symbol] =−μ(du/dy), where μ is viscosity and u is velocity of the fluid at a particular depth in the channel. Knowing media viscosity and fluid velocity, applied shear stress can be calculated.

Cell populations that retain the ability to differentiate into multiple cell types (e.g., stem cells) have proven useful for developing large numbers of lineage specific differentiated cell populations. Mesenchymal stem cells (MSCs) are one such type of stem cell and are known for being both multipotent and self-renewing. MSCs have thus emerged as candidate cellular therapeutics and can potentially provide a sustained source of bioactive immunomodulatory molecules. However, presently one of the obstacles limiting clinical efficacy of use of MSC is that the induction of MSC function is heavily dependent upon the presence of cytokines and signals produced by activated immune cells that in turn initiate the immunomodulatory activities of MSC. Prior to the present methods, for example, this variability has translated into unpredictable therapeutic activity of stem cell compositions.

In some aspects, a bioreactor system described herein provides increased numbers of stem cells (e.g., MSCs), which have also been predictably and reliably conditioned in vitro with regards to immunomodulatory function. Stem cells, thus obtained, provide therapeutically effective numbers of MSC which are reliably and consistently conditioned with regards to immunomodulatory function. In other embodiments, the system provides a source of secreted factors which can be isolated and purified for use as therapeutics.

In some embodiments, such methods can provide conditioned cells, such as MSC, for use in therapy and, for example, suppression of chronic or acute inflammation associated with injury, graft-versus-host disease, and autoimmunity.

Certain embodiments include a system for use in producing increased numbers of therapeutically predictably conditioned cells such as, but not limited to, MSCs for use in cell-based therapies. In some embodiments, the system described can be used for, among other things, conditioning MSCs to exhibit anti-inflammatory and immunomodulatory properties, to treat many types of musculoskeletal trauma and inflammatory conditions when, for example, such conditioned cells or factors they produce are injected at the site of an injury.

In some embodiments, this system comprises a modular bioreactor system that integrates control of the hydrodynamic microenvironment to direct mechanical-based conditioning of cells, such as, but not limited to MSC.

A bioreactor apparatus can be used to condition cells within a stream of media fluid in accordance with embodiments of the present invention (e.g., the media is passed over adherent cells to provide an applied shear force). Embodiments of such apparatus are illustrated in the appended figures, which are discussed below.

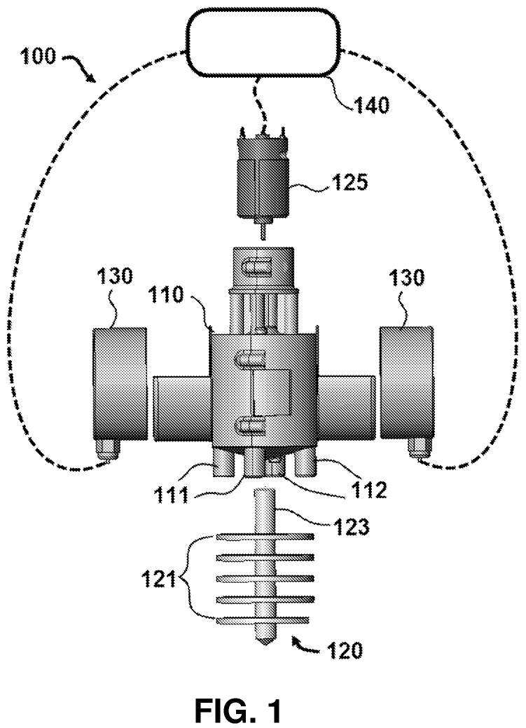

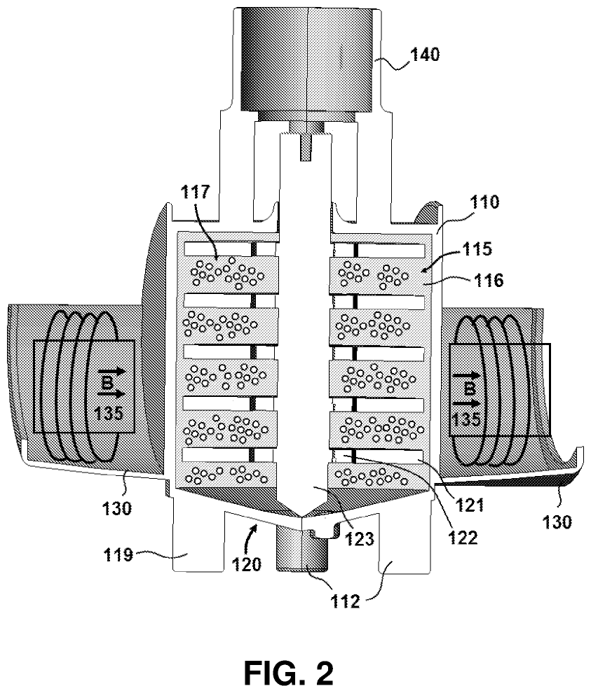

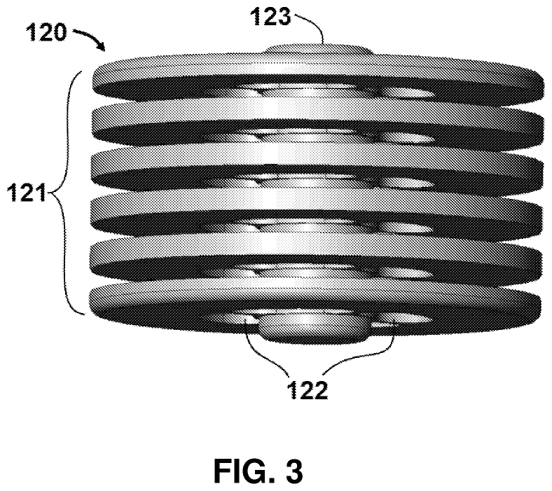

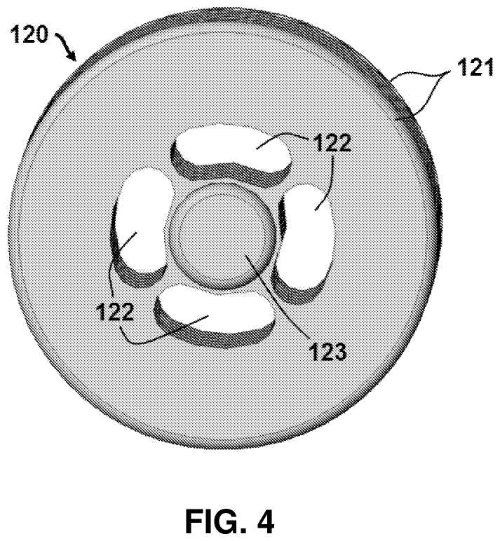

Referring initially to FIGS. 1-4, exploded and partial section views of an apparatus 100 are shown. In this embodiment, apparatus 100 comprises a stationary member 110, a rotating member 120 and a magnetic field generator 130. As used herein, “magnetic field generator” includes any device capable of generating a magnetic field, including for example, a permanent magnet, an electromagnetic, a solenoid coil, etc.

In addition, apparatus 100 comprises a cell culture medium 115 with magnetic beads 117 in a fluid 116, where cell culture medium 115 is contained within stationary member 110 and proximal to rotating member 120. In the embodiment shown, stationary member 110 comprises one or more inlets 119 and one or more outlets 112 to circulate culture medium 115. Apparatus 100 further comprises a controller 140 configured to control magnetic generator 130 and/or rotating member 120.

As explained in further detail below, during operation of apparatus 100 magnetic generator 130 can generate a variable magnetic field 135 on magnetic beads 117. The embodiment shown in FIGS. 1-4 illustrates a configuration with magnetic generator 130 generating a magnetic field 135 that is acting upon magnetic beads 117 with a force in a horizontal direction from left to right. However, it is understood that other embodiments may be configured such that magnetic generator 130 generates magnetic field 135 in a different orientation. For example, in other embodiments, magnetic generator 130 may generate magnetic field 135 in a vertical direction such that the magnetic force acting upon magnetic beads 117 is acting to counteract the force of gravity (e.g. in an upward direction). In still other embodiments, magnetic generator 130 may generate magnetic field 135 in in other directions, including for example, at an angle to the axis of rotation of rotating member 120, a downward direction, or other direction as desired.

During operation of apparatus 100, rotating member 120 rotates within stationary member 110. This rotational movement of rotating member 120 imparts a rotational shear force on cell culture medium 115 due to the viscosity of cell culture medium 115 and the surface tension between cell culture medium 115 and the surfaces of rotating member 120 and stationary member 110. However, the cells of cell culture medium 115 that are not proximal to a surface of either rotating member 120 or stationary member 110 may see reduced shear stress, dependent upon the viscosity and surface tension of cell culture medium 115. In the volumes of cell culture medium 115 that are not proximal to a surface, the lack of differential velocity in the cell culture medium 115 could lead to reduced shear stress.

Accordingly, apparatus 100 provides for increased shear force on cells in cell culture medium 115 by generating magnetic forces that act upon magnetic beads 117. These magnetic forces act upon magnetic beads 117 in addition to the rotational forces applied by rotating member 120. As used herein, the term “magnetic beads” includes various types of magnetic beads, including for example, paramagnetic beads. Paramagnetic beads do not retain residual magnetism and can provide advantages in certain applications over ferromagnetic beads. For example, the ability of paramagnetic beads to resist residual magnetism can reduce the likelihood of the beads clumping together, which would reduce the surface area of the beads available to the cells.

In exemplary embodiments, the cells in cell culture medium 115 are distributed on the surface of magnetic beads 117 to provide increased surface area for cell generation. However, if a magnetic force were not applied to magnetic beads 117, the rotary shear force applied to magnetic beads 117 by rotating member 120 would be reduced as magnetic beads 117 moved in the direction of fluid flow created by the rotating member 120.

In exemplary embodiments of the present disclosure, apparatus 100 can counteract the rotational fluid forces acting on magnetic beads 117 by pulsing magnetic field 135 such that the magnetic force acting on magnetic beads 117 counteracts or overcomes the rotational fluid forces generated by rotating member 120. For example, magnetic field 135 can be quickly pulsed to generate a force in one direction that is stronger than the rotational fluid force acting on magnetic beads 117 (where the rotational fluid force is acting on magnetic beads in a different direction than the magnetic force). Such action will cause magnetic beads 117 to move in the direction of the force generated by magnetic field 135. Magnetic field 135 can then be reversed (e.g. by changing the direction of current in a coil) so that the magnetic force acting on magnetic beads 117 is acting in the opposite direction of the original magnetic force. Magnetic field 135 can be quickly pulsed in short durations by controller 140 in opposite directions such that magnetic beads 117 are essentially stationary while cell culture medium 115 is rotated by rotating member. Accordingly, the fluid shear stress on cells located on the surface of magnetic beads 117 is increased due to the increase in relative velocity between the surface of magnetic beads 117 and cell culture medium 115. As discussed elsewhere in this disclosure, the ability to condition the cells by providing controlled shear forces to the cells can provide substantial benefits.

In the embodiment shown, an electric motor 125 is used to rotate rotating member 120, which comprises a plurality of discs 121 with apertures 122 coupled to a shaft 123. It is understood rotating member 120 is one exemplary configuration, and that other embodiments may comprise a rotating member with a different configuration shown in FIGS. 1-4. Discs 121 provide increased surface area for cell generation, and during operation of apparatus 100 the shear force can be controlled by adjusting the rotational velocity of rotating member 120 as well as the magnetic force applied by magnetic generator 130.

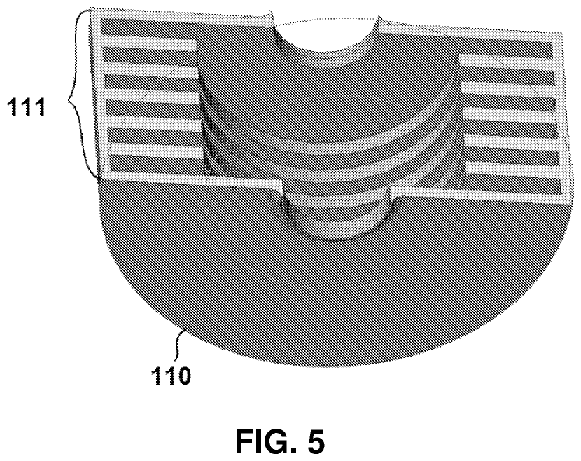

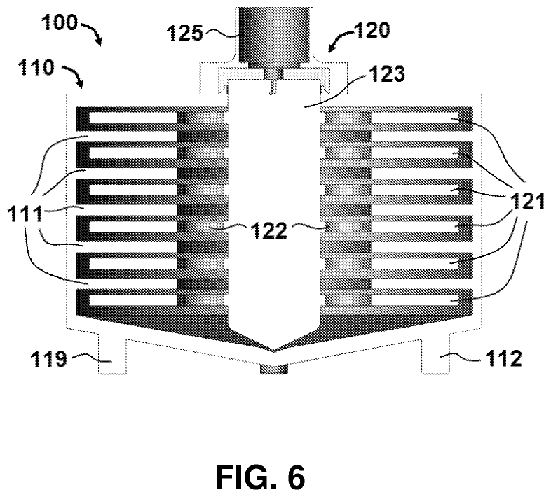

Referring now to FIG. 5, a perspective view of one portion of one embodiment of stationary member 110 is shown. In this embodiment, stationary member 110 comprises a plurality of annular surfaces 111 that are configured to interdigitate with discs 121, as shown in partial schematic view of FIG. 6. In certain embodiments, the overlap of discs 121 and annular surfaces 111 restricts the range of delivered shear stress (e.g. 7-10 dynes/cm² in specific examples). The gaps near the central portion allow circulation of the cell culture medium and keep the delivered shear from dropping below the desired range (to counteract the fact that shear decreases as the cells are located closer to the center).

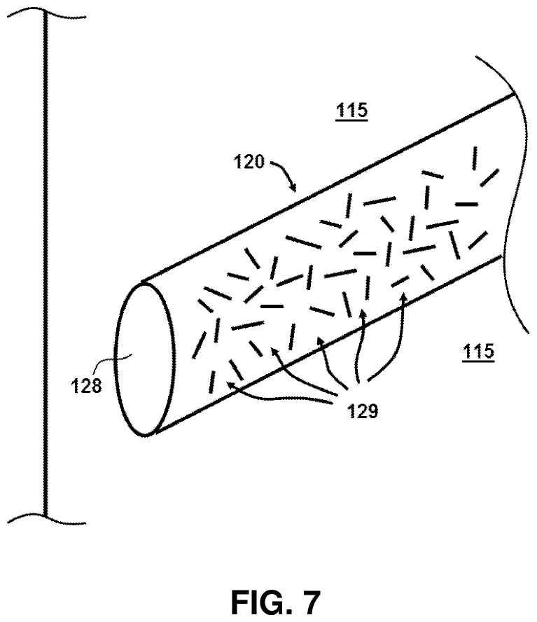

Referring now to FIG. 7, another embodiment of rotating member 120 is shown. In this embodiment, rotating member 120 is not configured as a rotor with a plurality of discs that are interdigitated with annular surfaces of a stator. Instead, rotating member 120 is configured as a cylindrical tube 128 comprising a mesh network of randomly oriented fibers 129 with diameter in the micron level range (e.g. between 10-100 microns, or more particularly approximately 50 microns).

During operation, cells could be cultured onto the mesh of randomly oriented fibers 129 and rotated through the fluid of the cell culture medium 115 (not shown in FIG. 7). The embodiment shown in FIG. 7 can be used in conjunction with a magnetic field generator as discussed elsewhere in this disclosure. The rotation of cylindrical tube and the application of the magnetic field can be used to control the shear stress applied to the cells. Still other embodiments could incorporate a cage of similar dimensions as the mesh tube described above, where the cage is designed to hold magnetic beads upon which the cells are cultured. In such embodiments, the cage can constrain the path of the beads through the fluid medium.

Referring now to FIG. 8, another embodiment of apparatus 100 comprises a stationary member 110 configured as a toroidal container 118, but does not include a rotating member disposed within stationary member 110. Although not visible in the view shown in FIG. 8, apparatus 100 comprises a cell culture medium comprising magnetic beads, as described in other embodiments in this disclosure. In addition, apparatus 100 comprises a magnetic generator 130 configured as a series of coils 131 wrapped around toroidal container 118. Controller 140 controls the operation of magnetic generator 130 to generate magnetic fields that act upon the magnetic beads of the cell culture medium to move the magnetic beads around toroidal container 118 and through the fluid of the cell culture medium. The cells on the surface of the magnetic beads are therefore subjected to shear stress as they move through the fluid of the cell culture medium. The shear stress can be controlled via the application of a magnetic field to the magnetic beads in the cell culture medium.

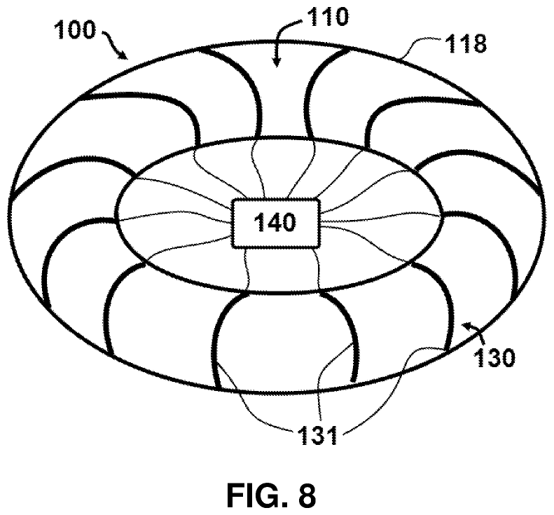

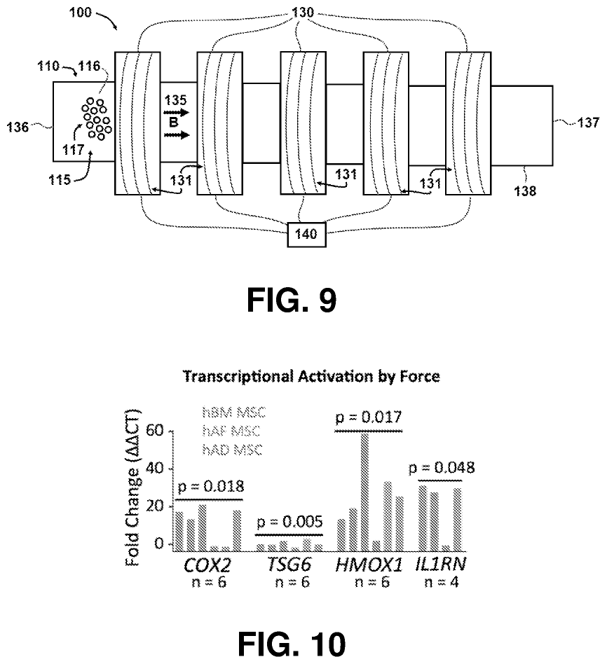

Referring now to FIG. 9 an embodiment of apparatus 100 is shown which comprises a stationary member 110 and a plurality of magnetic field generators 130. In the illustrated embodiment, stationary member 110 is configured as a linear tubular container 138 comprising a first end 136 and a second end 137, and magnetic field generators 130 are configured as a series of coils 131 wrapped around linear tubular container 138. In addition, apparatus 100 comprises a cell culture medium 115 with magnetic beads 117 in a fluid 116, where cell culture medium 115 is contained within stationary member 110.

In certain embodiments cell culture medium 115 is appropriate for the growth of stem cells, including for example, mesenchymal stem cells (MSCs). In particular embodiments, MSCs are deposited on the surface of magnetic beads 117 that are coated in a polymer suitable for cell adhesion, e.g. polystyrene or polycarbonate, and may further be coated with specific adhesion molecules, e.g. collagen or fibronectin. Examples of such beads are commercially available (e.g. MACS® Cell Separation System available from Miltenyi Biotec). Once the MSCs have been deposited onto the beads, they are placed into the tube with the growth medium

Apparatus 100 further comprises a controller 140 configured to activate magnetic generators 130 to generate a magnetic field 135 that acts upon magnetic beads 117 of the cell culture medium 115 to move magnetic beads 117 from first end 136 toward second end 137 (and/or vice versa) and through fluid 116 of cell culture medium 115. The cells on the surface of magnetic beads 117 are therefore subjected to shear stress as they move through fluid 116 of cell culture medium 115. The shear stress can be controlled via the application of magnetic field 135 to magnetic beads 117 in cell culture medium 115.

During operation of apparatus 100 magnetic generator 130 can generate a variable magnetic field 135 on magnetic beads 117. The embodiment shown in FIG. 9 illustrates a configuration with magnetic generator 130 generating a magnetic field 135 that is acting upon magnetic beads 117 with a force in a horizontal direction from left to right from first end 136 toward second end 137. However, it is understood that the illustrated embodiment may be operated such that magnetic generator 130 generates magnetic field 135 in a different orientation. For example, in other embodiments, magnetic generator 130 may generate magnetic field 135 in a direction such that the magnetic force acting upon magnetic beads 117 is acting in a horizontal direction from right to left from second end 137 toward first end 136, or other direction as desired.

In certain embodiments, controller 140 can sequentially activate and deactivate magnetic generators 130 by controlling the electrical current provided to adjacent coils 131. For example, controller 140 can provide electrical current to coils 131 in the leftmost magnetic generator 130, which will pull magnetic beads 117 to the center of the coil, at which point the coil is turned off and the next adjacent coil energized, pulling magnetic beads 117 along linear tubular container 138. In this manner, magnetic beads 117 are accelerated along the length of linear tubular container 138 and moved within linear tubular container 138. This creates shear for the cells on the surface of magnetic beads 117 as they flow through fluid 116. By altering the current and coil timing, specific patterns of fluid shear stress can be achieved in the cells. Once the cells have reached the opposite end of linear tubular container 138, the process can be reversed to drive magnetic beads 117 in the opposite direction (e.g. away from second end 137 and toward first end 136). Accordingly, apparatus 100 provides for increased shear force on cells in cell culture medium 115 by generating magnetic forces that act upon magnetic beads 117.

In certain embodiments, modular cell preparation apparatus as disclosed herein can be operated to control the hydrodynamic microenvironment to direct mechanotransduction conditioning of cells such as, but not limited to MSC, in a controlled manner By way of example, the studies provided herein demonstrate that the methods and apparatus described condition cell populations, including for example MSC, by subjecting them to a uniform and controllable shear stress as needed to condition such cells to express a particular activity, including, but not limited to, the induction and release of immunomodulatory factors.

The studies provided in the examples below demonstrate methods that human cell cultures, for example MSC, subject to shear stress of the type similar to that provided by the present apparatus, albeit using a less flexible system, using a device that is far more limited and less preparative in the scale of its abilities to administer shear stress. However, these studies illustrate that shear stress can be used to condition cells to express a particular activity, such as, but not limited to the induction and release of immunomodulatory factors.

The results presented herein demonstrate that functional MSCs can be directly conditioned to express and produce anti-inflammatory and immunomodulatory factors. In the context of cellular therapy, this technique promises to provide relief to patients affected by or at risk for inflammation associated with injury or disease. This indicates that conditioning of MSCs using shear stress of the type provided by the present system substantially increases their ability to inhibit inflammatory cells in pre-existing inflammatory environments and may aid in the prevention and resolution of inflammation.

Moreover, by using the system described herein, conditioning can be completed more rapidly, uniformly and reliably than when using alternatively available techniques of inducing MSC cell immunomodulatory activity, including for example, the production of anti-inflammatory molecules. The system described to condition cells can be particularly advantageous when a subject's own (autologous) cells are to be used as a therapeutic and a method for cell expansion and conditioning is required.

In the absence of conditioning, naive MSCs express little to none of the key mediators of immunosuppression such as the multifunctional anti-inflammatory proteins TNF-α stimulated protein 6 (TSG-6), prostaglandin E2 (PGE2), and interleukin (IL)-1 receptor antagonist (IL1RN). As detailed in the examples below, MSCs derived from three human tissue sources, bone marrow, adipose, and amniotic fluid, were all found to be responsive to this system of shear stress-based conditioning such that activation of immunomodulatory signaling was detectable to varying extents. Specifically, evaluation of conditioned human bone marrow-derived MSCs, using laminar shear stress of the type provided by the present system stimulated profound up-regulation of gene expression with from 6- to 120-fold increases, in the transcription of MSC genes encoding TSG-6, COX-2, IL1Ra, HMOX-1, LIF, and KLF2.

Exemplary embodiments include methods for providing a population of conditioned cells, the method comprising: obtaining a population of cells and subjecting the cells to a controlled shear stress. Certain embodiments include methods for providing a population of conditioned cells, the method comprising: obtaining a population of cells; culturing said cells in a cell media; and subjecting the cells to a controllable shear stress of sufficient force to condition the cells. In some embodiments, the cells are originally obtained from a mammal. In some embodiments, the cells are originally obtained from a companion animal. In preferred embodiments, the cells are originally obtained from a human. In some embodiments, the cells are originally obtained from bone marrow. In some embodiments, the cells are originally obtained from amniotic fluid while in other embodiments. While in some embodiments, the cells are originally obtained from adipose tissue. In some embodiments, the cells subjected to a controlled shear stress are MSC.

In additional embodiments, are methods of obtaining a therapeutically effective number of conditioned cells. In some embodiments, are methods of obtaining a therapeutically effective number of cells conditioned using the apparatus and methods described herein. In some embodiments, are methods of obtaining a therapeutically effective number of cells conditioned using the method comprising: obtaining a population of cells; applying a controlled shear stress of sufficient force to condition such cells to act as desired. In some other embodiments, are methods of obtaining a therapeutically effective number of cells conditioned using the method comprising: obtaining a population of cells; applying a controlled shear stress of sufficient force to condition such cells to act as desired. In some embodiments are methods of obtaining a therapeutically effective number of cells conditioned using the method comprising: obtaining a population of cells; culturing the cells on a first culture surface in a cell media, such that the cells adhere to on the first culture surface; and applying a controlled shear stress of sufficient force to condition such cells to act as desired. In some embodiments are methods of obtaining a therapeutically effective number of cells conditioned using the method comprising: obtaining a population of cells; culturing the cells on a culture surface in a cell media, such that the cells adhere to on the barrier; and applying a controlled shear stress of sufficient force to condition such cells to act as desired.

In similar embodiments, are methods for providing a population of conditioned cells comprising: obtaining a population of cells; culturing the cells in a culture system in a cell media, such that the cells adhere to the barrier; passing the cell media over said cells to provide a controlled fluid laminar shear stress of sufficient force to condition said cells. In some embodiments, the conditioned cells express anti-inflammatory activity. In some embodiments, the anti-inflammatory activity includes increased expression of genes selected from a group comprising those that encode TSG-6, COX-2, IL1RN, HMOX-1, LIF, or KLF2. In some embodiments, the activity includes increased expression of COX2 protein by the conditioned cells.

Additional embodiments include compositions comprising cells that have been conditioned using controlled shear stress in the apparatus described in claims 1-10. Some embodiments include compositions comprising cells that have been conditioned using a method comprising: obtaining a population of cells; culturing said cells in a culture system in a cell media, such that the cells adhere to the barrier; and applying a fluid laminar shear stress of sufficient force to condition said cells. In similar embodiments, are compositions comprising cells that have been conditioned using a method for providing a population of conditioned cells comprising: obtaining a population of cells; culturing the cells in a culture system in a cell media, such that the cells adhere to the barrier; passing said cell media over said cells to provide a fluid laminar shear stress of sufficient force to condition said cells. In some embodiments, the compositions comprise conditioned cells that express anti-inflammatory activity. In some embodiments, the anti-inflammatory activity includes increased expression of genes selected from a group comprising those that encode TSG-6, COX-2, IL1RN, HMOX-1, LIF, or KLF2. In some embodiments, the compositions comprise conditioned cells that express increased levels of COX2 protein. In additional embodiments the described device and methods may be used to stimulate the expression and release of anti-inflammatory factors, which can be isolated from the media and be used as therapeutics.

In additional embodiments are methods of treating a subject in need of such a treatment with cells conditioned by the methods described. In alternative embodiments are methods of treating a subject in need of such a treatment may include factors released by cells conditioned using the described methods.

In some embodiments, methods of treating a subject include but are not limited to, obtaining a population of conditioned cells produced in accordance with the described system and administering the cells to the subject in need of treatment. In some embodiments, the subject is in need of an anti-inflammatory therapy and the population of cells are human MSC whose anti-inflammatory activity has been induced using the described system. In some embodiments, a therapeutic dose of such cells may comprise at least 1×10², 1×10³, 1×10⁴, 1×10⁵ or 1×10⁶ cells which are introduced into the subject in need of therapy. In some embodiments anti-inflammatory activity of the conditioned cell population can be used to treat acute disorders such as, but not limited to, muscular skeletal injuries such as orthopedic or spinal cord injury or traumatic brain injury.

Cell culture conditioning systems are described in various embodiments herein and it is appreciated that additional methods for the culture and maintenance of cells, as would be known to one of skill, may be used with the present embodiments. In certain embodiments, for culture, various matrix components may be used in culturing, maintaining, or differentiating human stem cells. In addition to those described in the examples below, for example, collagen IV, fibronectin, laminin, and vitronectin in combination may be used to coat a culturing surface as a means of providing a solid support for pluripotent cell growth. Matrigel™ may also be used to provide a substrate for cell culture and maintenance of human pluripotent stem cells. Matrigel™ is a gelatinous protein mixture secreted by mouse tumor cells and is commercially available from BD Biosciences (New Jersey, USA). This mixture resembles the complex extracellular environment found in many tissues and is used by cell biologists as a substrate for cell culture.

In some embodiments of cell culturing, once a culture container is full (e.g., confluent), the colony is split into aggregated cells or even single cells by any method suitable for dissociation, which cells are then placed into new culture containers for passaging. Cell passaging or splitting is a technique that enables cells to survive and grow under cultured conditions for extended periods of time. Cells typically would be passaged when they are about 70%-100% confluent.

In certain aspects, starting cells for the present conditioning system may comprise at least or about 10⁴, 10⁵, 10⁶, 10⁷, 10⁸, 10⁹, 10¹⁰, 10¹¹, 10¹², 10¹³ cells or any range derivable therein. The starting cell population may have a seeding density of at least or about 10, 10¹, 10², 10³, 10⁴, 10⁵, 10⁶, 10⁷, 10⁸ cells/mL, or any range derivable therein.

As the basal medium, in addition to that described in the examples below, a range of media is available including defined medium, such as Eagle's Basal Medium (BME), BGJb, CMRL 1066, Glasgow MEM, Improved MEM Zinc Option, Iscove's modified Dulbecco's medium (IMDM), Medium 199, Eagle MEM, αMEM, DMEM, Ham, RPMI 1640, and Fischer's media. Additional examples of media that may be used according to the embodiments include, without limitation, Lonza Therapeak (chemically defined) medium, Irvine Scientific Prime-XV (SFM or XSFM), PromoCell MSC Growth Medium (DXF), StemCell Technologies Mesencult (ACF), or human platelet or platelet-lysate enriched medium.

In further embodiments, the media can also contain supplements such as B-27 supplement, an insulin, transferrin, and selenium (ITS) supplement, L-Glutamine, NEAA (non-essential amino acids), P/S (penicillin/streptomycin), N2 supplement (5 μg/mL insulin, 100 μg/mL transferrin, 20 nM progesterone, 30 nM selenium, 100 μM putrescine and β-mercaptoethanol (β-ME). It is contemplated that additional factors may or may not be added, including, but not limited to fibronectin, laminin, heparin, heparin sulfate, retinoic acid.

Additional factors may be added to a media for use in conjunction with sheer stress for generating a conditioned composition, such as a population of conditioned cells. Thus, in some embodiments, at least one chemical modulator of hematopoiesis may be applied before, during, or after biomechanical stimulation. Examples of additional components that could be added to media include, without limitation, Atenolol, Digoxin, Doxazosin, Doxycycline, Fendiline, Hydralazine, 13-hydroxyoctadecadienoic acid (13(s)-HODE), Lanatoside C, NG-monomethyl-L-arginine (L-NMMA), Metoprolol, Nerifolin, Nicardipine, Nifedipine, Nitric oxide (NO) or NO signaling pathway agonists, 1H-[1,2,4]oxadiazolo-[4,3-a]quinoxalin-1-one (ODQ), Peruvoside, Pindolol, Pronethalol, Synaptosomal protein (SNAP), Sodium Nitroprusside, Strophanthidin, Todralazine, 1,5-Pentamethylenetetrazole, Prostaglandin E 2 (PGE2), PGE2 methyl ester, PGE2 serinol amide, 11-deoxy-16,16-dimethyl PGE2, 15(R)-15-methyl PGE2, 15(S)-15-methyl PGE2, 6,16-dimethyl PGE2, 16,16-dimethyl PGE2 p-(p-acetamidobenzamido) phenyl ester, 16-phenyl tetranor PGE2, 19(R)-hydroxy PGE2, Prostaglandin B2, Prostacyclin (PGI2, epoprostenol), 4-Aminopyridine, 8-bromo-cAMP, 9-deoxy-9-methylene PGE2, 9-deoxy-9-methylene-16,16-dimethyl PGE2, a PGE2 receptor agonist, Bapta-AM, Benfotiamine, Bicuclline, (2′Z,3′E)-6-Bromoindirubin-3′-oxime (BIO), Bradykinin, Butaprost, CaylO397, Chlorotrianisene, Chlorpropamide, Diazoxide, Eicosatrienoic Acid, Epoxyeicosatrienoic Acid, Flurandrenolide, Forskolin, Gaboxadol, Gallamine, Indanyloxyacetic acid 94 (IAA 94), Imipramine, Kynurenic Acid, L-Arginine, Linoleic Acid, LY171883, Mead Acid, Mebeverine, 12 Methoxydodecenoic acid, N-Formyl-Met-Leu-Phe, Prostaglandin E2 receptor EP2-selective agonist (ONO-AE1-259), Peruvoside, Pimozide, Pindolol, Sodium Nitroprusside, Sodium Vanadate, Strophanthidin, Sulprostone, Thiabendazole, Vesamicol, 1,2-Didecanoyl-glycerol (10:0), 11,12 Epoxyeicosatrienoic acid,1-Hexadecyl-2-arachidonoyl-glycerol, 5-Hydroxydecanoate, 6-Formylindolo [3,2-B] carbazole, Anandamide (20:3,n-6), Carbacyclin, Carbamyl-Platelet-activating factor (C-PAF), or S-Farnesyl-L-cysteine methyl ester.

In further aspects, a media can include one or more growth factors, such as members of the epidermal growth factor family, e.g., EGF, members of the fibroblast growth factor family (FGFs) including FGF2 and/or FGF8, members of the platelet derived growth factor family (PDGFs), transforming growth factor (TGF)/bone morphogenetic protein (BMP) factor family antagonists including but not limited to noggin, follistatin, chordin, gremlin, cerberus/DAN family proteins, ventropin amnionless, TGF, BMP, and GDF antagonists could also be added in the form of TGF, BMP, and GDF receptor-Fc chimeras. Other factors that may or may not be added include molecules that can activate or inactivate signaling through Notch receptor family, including but not limited to proteins of the Delta-like and Jagged families as well as gamma secretase inhibitors and other inhibitors of Notch processing or cleavage such as DAPT. Additional growth factors may include members of the insulin like growth factor family (IGF), the wingless related (WNT) factor family, and the hedgehog factor family.

In still further aspects, a media can include one or more priming agents such as an inflammatory cytokine, LPS, PHA, Poly I:C, and/or ConA. Additional priming agents that may be used according the embodiments include those detailed Wagner et al., 2009, which is incorporated herein by reference.

The medium can be a serum-containing or serum-free medium. The serum-free medium may refer to a medium with no unprocessed or unpurified serum and accordingly, can include media with purified blood-derived components or animal tissue-derived components (such as growth factors). From the aspect of preventing contamination with heterogeneous animal-derived components, serum can be derived from the same animal as that of the cell(s).

The medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thiolglycerol, or equivalents thereto. The alternatives to serum can be prepared by the method disclosed in International Publication No. WO98/30679, for example. Alternatively, any commercially available materials can be used for more convenience. The commercially available materials include knockout Serum Replacement (KSR), Chemically-defined Lipid concentrated (Gibco), and Glutamax (Gibco).

The medium can also contain fatty acids or lipids, amino acids (such as non-essential amino acids), vitamin(s), growth factors, cytokines, antioxidant substances, 2-mercaptoethanol, pyruvic acid, buffering agents, and inorganic salts. The concentration of 2-mercaptoethanol can be, for example, about 0.05 to 1.0 mM, and particularly about 0.1 to 0.5, or 0.01, 0.02, 0.03, 0.04, 0.05, 0.1, 0.2, 0.5, 0.8, 1, 1.5, 2, 2.5, 5, 7.5, 10 mM or any intermediate values, but the concentration is particularly not limited thereto as long as it is appropriate for culturing the stem cell(s).

The cells may be cultured in a volume of at least or about 0.005, 0.010, 0.015, 0.2, 0.5, 1, 2, 5, 10, 20, 30, 40, 50, 100, 150, 200, 250, 300, 350, 400, 450, 500, 550, 600, 800, 1000, 1500 mL, or any range derivable therein, depending on the needs of the culture. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture surface and chamber formed between the wall 13 of the feed cap 12 and the barrier 39 of the intermediate module 30 when the apparatus is assembled can be prepared with cellular adhesive or not depending upon the purpose. The cellular adhesive culture vessel can be coated with a suitable substrate for cell adhesion (e.g. extracellular matrix [ECM]) to improve the adhesiveness of the vessel surface to the cells. The substrate used for cell adhesion can be any material intended to attach stem cells or feeder cells (if used). Non-limiting substrates for cell adhesion include collagen, gelatin, poly-L-lysine, poly-D-lysine, poly-D-ornithine, laminin, vitronectin, and fibronectin and mixtures thereof, for example, protein mixtures from Engelbreth-Holm-Swarm mouse sarcoma cells (such as Matrigel™ or Geltrex) and lysed cell membrane preparations. In specific embodiments culture includes a matrix comprising poly-L-lysine (or poly-D-lysine) and laminin.

Other culturing conditions can be appropriately defined. For example, the culturing temperature can be about 30 to 40° C., for example, at least or about 31, 32, 33, 34, 35, 36, 37, 38, 39° C. but particularly not limited to them. The CO₂ concentration can be about 1 to 10%, for example, about 2 to 7%, or any range derivable therein. The oxygen tension can be at least or about 1, 5, 8, 10, 20%, or any range derivable therein.

Essentially free of an “externally added” component refers to a medium that does not have, or that have essentially none of, the specified component from a source other than the cells in the medium. “Essentially free” of externally added growth factors or polypeptides, such as FGF or EGF etc., may mean a minimal amount or an undetectable amount of the externally added component. For example, a medium or environment essentially free of FGF or EGF polypeptide can contain less than 1, 0.9, 0.8, 0.7, 0.6, 0.5, 0.4, 0.3, 0.2, 0.1, 0.01, 0.001 ng/mL or any range derivable therein.

In some embodiments, cells conditioned using the system described have a variety of therapeutic uses. In particular, if the cells are human MSCs, diseases or disorders for which such conditioned cells can be used therapeutically, or alternatively those diseases or disorders for which therapy with the factors produced and isolated from cultured cells, including but not limited to MSCs subjected to conditioning with the system described include, but are not limited to, autoimmune disorders (including but not limited to Rheumatoid Arthritis (RA), Systemic Lupus Erythematosis (SLE)), graft-versus-host disease, Crohn's disease, inflammatory bowel disease, neurodegenerative disorders, neuronal dysfunctions, disorders of the brain, disorders of the central nervous system, disorders of the peripheral nervous system, neurological conditions, disorders of memory and learning, cardiac arrhythmias, Parkinson's disease, ocular disorders, spinal cord injury, disorders requiring neural healing and regeneration, Multiple Sclerosis (MS), Amyelotrophic Lateral Sclerosis (ALS), Parkinson's disease, stroke, chronic or acute injury, bone repair, traumatic brain injury, orthopedic and spinal conditions, cartilage skeletal or muscular disorders, osteoarthritis, osteonecrosis, cardiovascular diseases, blood vessel damage linked to heart attacks or diseases such as critical limb ischemia, peripheral artery disease, atherosclerosis, and those benefiting from neovascularization, wounds, burns and ulcers.

In certain embodiments the presently disclosed system can be applied to condition cells and improve their immune regulatory properties. In certain embodiments, such compositions can be administered in combination with one or more additional compounds or agents (“additional active agents”) for the treatment, management, and/or prevention of among other things autoimmune diseases and disorders. Such therapies can be administered to a patient at therapeutically effective doses to treat or ameliorate, among other things, immunoregulatory disease or disorders.

Toxicity and therapeutic efficacy of such conditioned cell or factor compositions can be determined by standard pharmaceutical procedures, using for example, cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, expressed as the ratio LD₅₀/ED₅₀. Compositions that exhibit large therapeutic indices are preferred. Compounds that exhibit toxic side effects may be used in certain embodiments, however, care should usually be taken to design delivery systems that target such compositions preferentially to the site of affected tissue, in order to minimize potential damage to unaffected cells and, thereby, reduce side effects.

Data obtained from cell culture assays and animal studies can be used in formulating a range of dosages for use in humans. The dosages of such compositions lie preferably within a range of circulating concentrations that include the ED₅₀ with little or no toxicity. The dosage may vary within this range depending on the dosage form employed and the route of administration utilized. For any composition, the therapeutically effective dose may be estimated initially from cell culture assays. A dose may be formulated in animal models to achieve a circulating plasma concentration range that includes the IC₅₀ (i.e., the concentration of the test composition that achieves a half-maximal inhibition of symptoms) as determined in cell culture. Such information can be used to more accurately determine useful doses in humans. Plasma levels may be measured, for example, by high performance liquid chromatography.

When the therapeutic treatment of among other things autoimmune disorders is contemplated, the appropriate dosage may also be determined using animal studies to determine the maximal tolerable dose, or MTD, of a bioactive agent per kilogram weight of the test subject. In general, at least one animal species tested is mammalian Those skilled in the art regularly extrapolate doses for efficacy and avoiding toxicity to other species, including human. Before human studies of efficacy are undertaken, Phase I clinical studies will help establish safe doses.

Additionally, the bioactive agent may be coupled or complexed with a variety of well-established compositions or structures that, for instance, enhance the stability of the bioactive agent, or otherwise enhance its pharmacological properties (e.g., increase in vivo half-life, reduce toxicity, etc.).

Cells conditioned using the present system or factors released from such cells and other such therapeutic agents can be administered by any number of methods known to those of ordinary skill in the art including, but not limited to, cell insertion during surgery, intravenous (I.V.), intraperitoneal (I.P.), intramuscular (I.M.), or intrathecal injection, inhalation, subcutaneous (sub-q), or topically applied (transderm, ointments, creams, salves, eye drops, and the like).

The following examples section provides further details regarding examples of various embodiments. It should be appreciated by those of skill in the art that the techniques disclosed in the examples that follow represent techniques and/or compositions discovered by the inventors to function well. However, those of skill in the art should, in light of the present disclosure, appreciate that many changes can be made in the specific embodiments which are disclosed and still obtain a like or similar result without departing from the spirit and scope of the invention. These examples are illustrations of the methods and systems described herein and are not intended to limit the scope of the invention. Non-limiting examples of such include, but are not limited to, those presented below.

As used herein, and unless otherwise indicated, the terms “treat,” “treating,” “treatment” and “therapy” contemplate an action that occurs while a patient is suffering from a disease or disorder that reduces the severity of one or more symptoms or effects of such disease or disorder. Where the context allows, the terms “treat,” “treating,” and “treatment” also refers to actions taken toward ensuring that individuals at increased risk of a disease or disorder, are able to receive appropriate surgical and/or other medical intervention prior to onset of a disease or disorder. As used herein, and unless otherwise indicated, the terms “prevent,” “preventing,” and “prevention” contemplate an action that occurs before a patient begins to suffer from a disease or disorder, that delays the onset of, and/or inhibits or reduces the severity of a disease or disorder.

As used herein, and unless otherwise indicated, the terms “manage,” “managing,” and “management” encompass preventing, delaying, or reducing the severity of a recurrence of a disease or disorder in a patient who has already suffered from such a disease, disorder or condition. The terms encompass modulating the threshold, development, and/or duration of a disease or disorder or changing how a patient responds to a disease or disorder.

As used herein, and unless otherwise specified, a “therapeutically effective amount” of cells, factor or compound is an amount sufficient to provide any therapeutic benefit in the treatment or management of a disease or disorder, or to delay or minimize one or more symptoms associated with a disease or disorder. A therapeutically effective amount means an amount of the cells, factor or compound, alone or in combination with one or more other therapies and/or therapeutic agents that provide any therapeutic benefit in the treatment or management of a disease or disorder. The term “therapeutically effective amount” can encompass an amount that alleviates a disease or disorder, improves or reduces a disease or disorder, improves overall therapy, or enhances the therapeutic efficacy of another therapeutic agent.

As used herein, and unless otherwise specified, a “prophylactically effective amount” of cells, factor or compound is an amount sufficient to prevent or delay the onset of a disease or disorder, or one or more symptoms associated with a disease or disorder, or prevents or delays its recurrence. A prophylactically effective amount of cells, factors or compound means an amount of the cells, factor or compound, alone or in combination with one or more other treatment and/or prophylactic agent that provides a prophylactic benefit in the prevention of a disease or disorder. The term “prophylactically effective amount” can encompass an amount of cells, factor or compound that prevents a disease or disorder, improves overall prophylaxis, or enhances the prophylactic efficacy of another prophylactic agent. The “prophylactically effective amount” can be prescribed prior to, for example, the disease or disorder.

As used herein, “patient” or “subject” includes mammalian organisms which are capable of suffering from a disease or disorder as described herein, such as human and non-human mammals, for example, but not limited to, rodents, mice, rats, non-human primates, companion animals such as dogs and cats as well as livestock, e.g., sheep, cow, horse, etc.

As used herein, “MSC” are Mesenchymal Stem Cells, such cells have also been referred to as Mesenchymal Stromal Cells.

As used herein, “controlled shear stress” refers to the ability to set the amount of shear stress applied to the cells by adjusting the flow rate of media across the surface. The stress is uniformly applied across the entire surface area of the plate.

As used herein, “conditioned cells” refers to cells which express additional functionality as a result of having been exposed to a shear stress.

As used herein “magnetic field generator” includes any device capable of generating a magnetic field, including for example, a permanent magnet, an electromagnetic, a solenoid coil, etc.

As used herein, the term “magnetic beads” includes various types of magnetic beads, including for example, paramagnetic beads. Paramagnetic beads do not retain residual magnetism and can provide advantages in certain applications over ferromagnetic beads.

The corresponding structures, materials, acts, and equivalents of all means or steps plus function elements in the claims below are intended to include any structure, material, or act for performing the function in combination with other claimed elements as specifically claimed. The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Without further elaboration, it is believed that one skilled in the art can, using the description herein, utilize the present methods to its fullest extent. The embodiments described herein are to be construed as illustrative and not as constraining the remainder of the disclosure in any way whatsoever. While preferred embodiments have been shown and described, many variations and modifications thereof can be made by one skilled in the art without departing from the spirit and teachings of the presently disclosed methods.

Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they are consistent with the present disclosure set forth herein.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

A pilot study was conducted to provide proof of principle that fluid shear stress can be used to condition stem cells, alter gene expression, and enhance functional activities. To exemplify the conditioning activity of shear stress of the type provided by the present system, but on a much smaller analytical scale, force (shear stress) was applied using custom fabricated slides or IBIDI® small-scale microfluidics channel slides obtained from IBIDI, LLC. (Verona, WI, USA). Human samples were harvested from bone marrow (BM), amniotic fluid (AF), or adipose (AD) tissue, processed for isolation and expansion of hMSC, and stored frozen. Frozen hMSCs were thawed and seeded into T225 cell culture flasks with 50 ml Minimum Essential Medium (MEM-α) (20% FBS, 5% Penicillin/Streptomycin, 5% Glutamine) The media was replaced every 3-4 days. hMSCs were maintained in culture until they were almost 100% confluent. The cells had a fibroblastic phenotype. Prior to seeding the cells onto the device (IBIDI® microfluidic channel slide or a custom fabricated slide) to provide fluid laminar shear stress similar to, but on a far more limed scale than that which is provided by the instant system, the culture surfaces were pre-coated with 100 ug/ml fibronectin in PBS for 30-45 min at 37° C. and washed 2× with PBS before seeding cells and allowed to sit in the incubator for 30-45 minutes while cells were prepared for seeding. Cultured hMSC were prepared by removing the media from the T225 flask using a vacuum and glass Pasteur pipette, the cells were washed 1× with room temperature PBS, which was removed by aspiration. 3 ml of a 0.25% trypsin solution was added and the flask was incubated at 37° C. for 5 min. Following this incubation, the flask was removed from the incubator and tapped vigorously to dislodge the cells. The flask was examined under a dissecting microscope to ensure that all of the cells had detached and were free-floating. At this point 9 ml of MEM-α was added to the flask and the total volume (12 ml) was removed and placed in a 15 ml conical tube. The tube was placed in a centrifuge and spun at 300 RCF for 5 minutes at room temperature. The supernatant was aspirated, leaving a small amount of media above the cell pellet, 3 ml MEM-α was added and the cell pellet was resuspended in this media. The number of live cells present was determined using trypan blue dye exclusion. Live cell counts were determined on a hemacytometer and the cells were resuspended to obtain the desired concentration for each assay (see Table 1). IBIDI channels were utilized to provide fluid laminar shear stress similar to, but less well controlled than, the type provided by the instant system. Cells were allowed to sit for 30-45 minutes before filling wells with 60 ul media per well (if too much time passes, the media will begin to evaporate from the channels). Cells were allowed to incubate for 12-18 hours. Tubing was then attached to the IBIDI® slide in the safety cabinet with a clean three way stopcock (all previously autoclaved or EtO sterilized, the three stop tubing required for use with a peristaltic pump may be EtO or UV sterilized) and then transferred to incubator. IBIDI channel experiments recirculated a total volume of 6 ml and large fabricated slide experiments recirculated 50 ml total volume. A peristaltic or Harvard syringe pump was programmed to push media across the culture surface at 15 dyne/cm2. Fluid shear stress was applied for 3, 6 or 8 hours.

TABLE 1

| | Cell | | Total | | Time of | |
|---|---|---|---|---|---|---|
| | Concentration | Culture | Volume | | Shear | |
| Assay | (cells/ml) | Platform | in channel | | Exposure | |
| qRT PCR | 3 × 10\hat{ }6 | IBIDI | 30 | ul | 3, 6, 8 | hours |
| Western Blot | 3 × 10\hat{ }6 | IBIDI | 30 | ul | 8 | hours |
| NF-kB Binding | 2 × 10\hat{ }6 | Custom | 10 | ml | 8 | hours |
| Assay | | | | | | |
| In Vivo Rat | 2 × 10\hat{ }6 | Custom | 10 | ml | 8 | hours |
| Experiments | | | | | | |
| ELISA assay | 2 × 10\hat{ }5 | IBIDI | 30 | ul | 3, 8 | hours |
| IF Staining | 2 × 10\hat{ }5 | IBIDI | 30 | ul | 3 | hours |

### Immunomodulatory Changes Due to Fluid Laminar Shear Stress

Naive MSCs do not express key mediators of immunosuppression, such as the multifunctional anti-inflammatory proteins such as TNF-α stimulated protein 6 (TSG-6), prostaglandin E2 (PGE2), and interleukin (IL)-1 receptor antagonist (IL1RN). MSCs derived from three human tissue sources, bone marrow, adipose, and amniotic fluid, were all found to be responsive to shear stress to varying extents. For example, a shear stress applied at a force of 15 dyne/cm² activated immunomodulatory signaling in MSCs collected from multiple human tissues. Evaluation of bone marrow-derived MSCs, laminar shear stress stimulated profound up-regulation, from 6- to 120-fold increases, in transcription of MSC genes encoding TSG-6, COX-2, IL1RN, HMOX-1, LIF, and KLF2. See for example (FIG. 10).

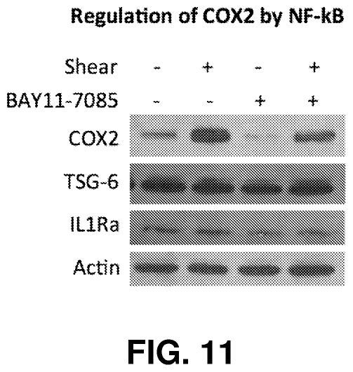

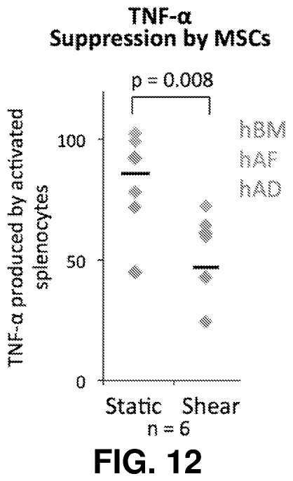

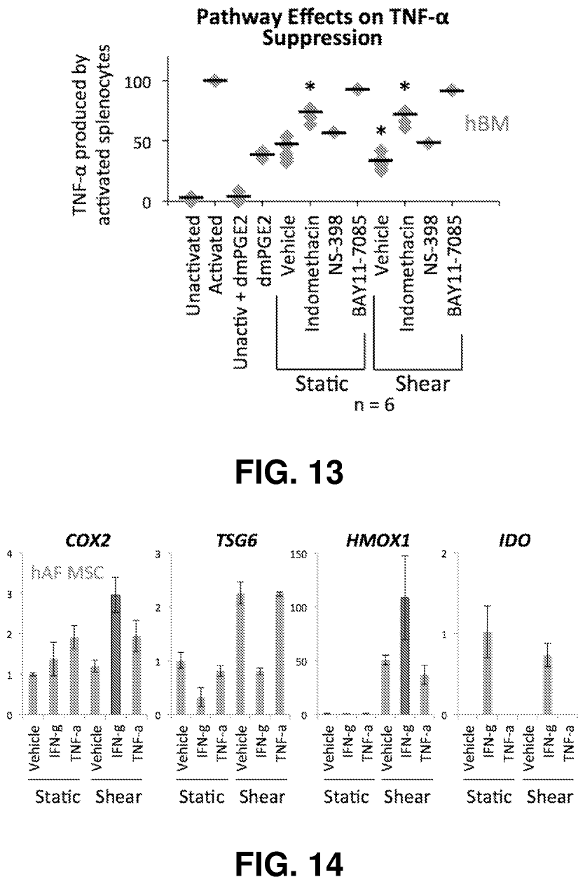

Similarly it was determined utilizing commercially available ELISAs that the media from human MSCs cultures that had been subject to fluid shear stress also contained immunomodulatory proteins, such as prostaglandin E2. In addition, Western blotting confirmed elevated protein levels (translation) of COX2, TSG6, and IL1RN. Actin protein expression levels, which are constant, were used as a control for baseline protein expression. In this study, it was determined that after exposure of human MSC (derived from hBM, human bone marrow MSC; hAF MSC, amniotic fluid MSC; hAD MSC, adipose-derived MSC) to 8 hours of fluid shear stress, there was a significant increase in the expression of COX2 protein as compared with media obtained from MSC that were not subject to fluid shear stress. It was also determined that this induction could be abrogated by the addition of 10 uM of the NF-kappa B antagonist BAY11-7085 (FIG. 11). Furthermore by utilizing commercially available ELISAs, it was determined that the media from human MSCs cultures that had been subject to fluid shear stress for as little as 3 hours had immunosuppressive activity, as evidenced by the 10-50% reduction in TNF-α when treated hMSCs were co-cultured with activated immune cells from the spleen (FIG. 12). It was also determined using cytokine suppression assays that application of COX or NF-kB inhibitors abrogated the ability of shear treated MSC to suppress TNF-αproduction; whereas, addition of a stabilized synthetic form of PGE2 (dmPGE2) reduced TNF-α to levels produced in the presence of sheared MSCs (FIG. 13). Additional evidence further suggests that sheared MSCs may be more responsive to other priming agents than MSC that have not been subject to a fluid shear stress, as a greater induction of COX2 and HMOX1 occurred with the addition of IFN-γ (FIG. 14). Thus, indicating that human MSC subject to fluid shear stress may act synergistically with cytokines when presented in, for example, combination therapies.

It was further determined that naive MSCs exposed to shear stress, with no preconditioning by inflammatory cytokines, were capable of blocking TNF-αsecretion by lipopolysaccharide (LPS)-activated mouse splenocytes (ranging from complete inhibition to 2-fold reduction below MSC cultured under static conditions, depending upon MSC donor and source variability).

### Neuroprotective Abilities:

Studies were performed to establish that cells such as MSC exposed to controlled shear stress can provide neuroprotection following, for example, traumatic brain injury (TBI). To do this a rat model was utilized to evaluate functional outcomes. Controlled cortical impact (CCI) in the rat presents morphologic and cerebrovascular injury responses that resemble human head trauma. Thus, characterization of the cellular and molecular alterations that potentiate neurological damage and inflammation provides a powerful tool to measure potential clinical efficacy of MSC preconditioning.

Twelve (12) rats were predicted to be necessary per condition to achieve 80% power, at an alpha error level of 0.05 (SAS predictive analytics software). Cells to be administered for cellular therapy were bone marrow-derived MSCs. MSCs were exposed to static conditions or to shear stress for 3 hrs at an intensity of 15 dyne/cm², a fluid flow rate and duration demonstrated to produce robust induction of COX2, TSG6, IL1RN, and HMOX1 and to suppress cytokine production in activated immune cells Immediately following application of force by the large capacity lateral flow system, 10×10⁶ cells/kg MSCs were transferred to recipient rats via tail vein injection (approximate dose of 2.5×10⁶ MSCs per rat).

Blood-brain barrier (BBB) permeability was determined using standard methods for examining leakage across the vasculature (utilizing dextran beads in suspension as described herein). Injury at the right parietal association cortex was introduced in male rats (225-250 grams) by a CCI device (Leica Impactor 1). In parallel, control rats were treated with CCI alone or simply anesthetized (sham control). Forty eight (48) hours after injury, the MSCs were administered. Twenty-four (24) hrs after the injection of MSCs, fluorescently conjugated Alexa 680-dextran beads (10 kDa, 0.5 ml of 1 mg/ml) were delivered via tail vein. Thirty (30) minutes after this dye was injected, animals were euthanized and perfused with 4% paraformaldehyde. Fixed brains were sectioned coronally at 1 mm thickness. Vascular leakage was measured by fluorescence intensity of brain sections in a LI-COR Odyssey CLx infrared laser scanner using 700 and 800 nm channels (800 nm signal was used for background subtraction). Histological analysis of the frequencies of certain immune and neural cell types that are known to be rapidly altered in response to neuroinflammation and are important indicators of prognosis. In future studies, in an independent cohort of rats, brain sections of between 8 to 50 um will be analyzed for inflammatory phenotypes in the CNS by immunohistochemistry using antibodies to detect microglia (Ibal, ED₁ or CD63), infiltrating neutrophils (RP-3), astroglia (GFAP), and neurons (NeuN), as well as an indicator of cell death (cleaved caspase 3). Staining of brain sections will be done using a standard free floating staining protocol or slide-mounted cryosections.

Cognitive recovery of treated and control rats can be assessed by a classic hippocampus-dependent spatial learning task, the Morris water maze, in which rats locate an underwater platform on the basis of extra-maze cues. For these studies, two (2) weeks following injury, learning is measured by speed, time spent in each quadrant, and distance of the path taken to find the platform. The same individuals will be tested at 4 weeks post injury for memory function by the same measures in the maze. The anticipated results are that delivery of sheared MSCs will reduce BBB permeability and inflammatory cell phenotypes in the brain relative to naïve static-cultured MSC and will also result in improved cognitive recovery.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

### REFERENCES

The following references, to the extent that they provide exemplary procedural or other details supplementary to those set forth herein, are specifically incorporated herein by reference.
1. English, K. (2013). Mechanisms of mesenchymal stromal cell immunomodulation. Immunology and cell biology 91(1): 19-26.
2. Krampera, M. (2011). Mesenchymal stromal cell ‘licensing’: a multistep process. Leukemia 25(9): 1408-1414.
3. Prockop, D. J. & Oh, J. Y. (2012). Mesenchymal stem/stromal cells (MSCs): role as guardians of inflammation. Molecular therapy: the journal of the American Society of Gene Therapy 20(1): 14-20.
4. Murphy, M. B., Moncivais, K. & Caplan, A. I. (2013). Mesenchymal stem cells: environmentally responsive therapeutics for regenerative medicine. Exp Mol Med 45: e54.
5. Wagner, J., Kean, T., Young, R., Dennis, J. E. & Caplan, A. I. (2009). Optimizing mesenchymal stem cell-based therapeutics. Curr Opin Biotechnol 20(5): 531-536.
6. Cleeland, C. S., Bennett, G. J., Dantzer, R., Dougherty, P. M., Dunn, A. J., Meyers, C. A., Miller, A. H., Payne, R., Reuben, J. M., Wang, X. S. & Lee, B. N. (2003). Are the symptoms of cancer and cancer treatment due to a shared biologic mechanism? A cytokine-immunologic model of cancer symptoms. Cancer 97(11): 2919-2925.
7. Kipnis, J., Gadani, S. & Derecki, N. C. (2012). Pro-cognitive properties of T cells. Nat Rev Immunol 12(9): 663-669.
8. Derecki, N. C., Cardani, A. N., Yang, C. H., Quinnies, K. M., Crihfield, A., Lynch, K. R. & Kipnis, J. (2010). Regulation of learning and memory by meningeal immunity: a key role for IL-4. J Exp Med 207(5): 1067-1080.
9. Spaggiari, G. M., Capobianco, A., Abdelrazik, H., Becchetti, F., Mingari, M. C. & Moretta, L. (2008). Mesenchymal stem cells inhibit natural killer-cell proliferation, cytotoxicity, and cytokine production: role of indoleamine 2,3-dioxygenase and prostaglandin E2. Blood 111(3): 1327-1333.
10. Nemeth, K., Leelahavanichkul, A., Yuen, P. S., Mayer, B., Parmelee, A., Doi, K., Robey, P. G., Leelahavanichkul, K., Koller, B. H., Brown, J. M., Hu, X., Jelinek, I., Star, R. A. & Mezey, E. (2009). Bone marrow stromal cells attenuate sepsis via prostaglandin E(2)-dependent reprogramming of host macrophages to increase their interleukin-10 production. Nature medicine 15(1): 42-49.
11. Prockop, D. J. (2013). Concise Review: Two negative feedback loops place mesenchymal stem/stromal cells at the center of early regulators of inflammation. Stem Cells 31(10): 2042-2046.
12. Yano, Y., Ozono, R., Oishi, Y., Kambe, M., Yoshizumi, M., Ishida, T., Omura, S., Oshima, T. & Igarashi, K. (2006). Genetic ablation of the transcription repressor Bachl leads to myocardial protection against ischemia/reperfusion in mice. Genes Cells 11(7): 791-803.
13. Richards, J. A., Wigmore, S. J. & Devey, L. R. (2010). Heme oxygenase system in hepatic ischemia-reperfusion injury. World J Gastroenterol 16(48): 6068-6078.
14. Decuypere, M. & Klimo, P., Jr. (2012). Spectrum of traumatic brain injury from mild to severe. The Surgical clinics of North America 92(4): 939-957, ix.
15. Shlosberg, D., Benifla, M., Kaufer, D. & Friedman, A. (2010). Blood-brain barrier breakdown as a therapeutic target in traumatic brain injury. Nat Rev Neurol 6(7): 393-403.
16. Kumar, A. & Loane, D. J. (2012). Neuroinflammation after traumatic brain injury: opportunities for therapeutic intervention. Brain, behavior, and immunity 26(8): 1191-1201.
17. Richardson, R. M., Singh, A., Sun, D., Fillmore, H. L., Dietrich, D. W., 3rd & Bullock, M. R. (2010). Stem cell biology in traumatic brain injury: effects of injury and strategies for repair. Journal of neurosurgery 112(5): 1125-1138.
18. Walker, P. A., Harting, M. T., Jimenez, F., Shah, S. K., Pati, S., Dash, P. K. & Cox, C. S., Jr. (2010). Direct intrathecal implantation of mesenchymal stromal cells leads to enhanced neuroprotection via an NFkappaB-mediated increase in interleukin-6 production. Stem cells and development 19(6): 867-876.
19. Pati, S., Khakoo, A. Y., Zhao, J., Jimenez, F., Gerber, M. H., Harting, M., Redell, J. B., Grill, R., Matsuo, Y., Guha, S., Cox, C. S., Reitz, M. S., Holcomb, J. B. & Dash, P. K. (2011). Human mesenchymal stem cells inhibit vascular permeability by modulating vascular endothelial cadherin/beta-catenin signaling. Stem cells and development 20(1): 89-101.
20. Walker, P. A., Shah, S. K., Jimenez, F., Aroom, K. R., Harting, M. T. & Cox, C. S., Jr. (2012). Bone marrow-derived stromal cell therapy for traumatic brain injury is neuroprotective via stimulation of non-neurologic organ systems. Surgery 152(5): 790-793.
21. Bonilla, C., Zurita, M., Otero, L., Aguayo, C., Rico, M. A., Rodriguez, A. & Vaquero, J. (2012). Failure of delayed intravenous administration of bone marrow stromal cells after traumatic brain injury. Journal of neurotrauma 29(2): 394-400.
22. Ramlackhansingh, A. F., Brooks, D. J., Greenwood, R. J., Bose, S. K., Turkheimer, F. E., Kinnunen, K. M., Gentleman, S., Heckemann, R. A., Gunanayagam, K., Gelosa, G. & Sharp, D. J. (2011). Inflammation after trauma: microglial activation and traumatic brain injury. Annals of neurology 70(3): 374-383.
23. Lee, H. J., Li, N., Evans, S. M., Diaz, M. F. & Wenzel, P. L. (2013). Biomechanical force in blood development: extrinsic physical cues drive pro-hematopoietic signaling. Differentiation; research in biological diversity 86(3): 92-103.
24. Adamo, L., Naveiras, O., Wenzel, P. L., McKinney-Freeman, S., Mack, P. J., Gracia-Sancho, J., Suchy-Dicey, A., Yoshimoto, M., Lensch, M. W., Yoder, M. C., Garcia-Cardena, G. & Daley, G. Q. (2009). Biomechanical forces promote embryonic haematopoiesis. Nature 459(7250): 1131-1135.
25. Adams, W. J., Zhang, Y., Cloutier, J., Kuchimanchi, P., Newton, G., Sehrawat, S., Aird, W. C., Mayadas, T. N., Luscinskas, F. W. & Garcia-Cardena, G. (2013). Functional vascular endothelium derived from human induced pluripotent stem cells. Stem cell reports 1(2): 105-113.
26. Daneman, R., Zhou, L., Kebede, A. A. & Barres, B. A. (2010). Pericytes are required for blood-brain barrier integrity during embryogenesis. Nature 468(7323): 562-566.
27. Bedi, S. S., Walker, P. A., Shah, S. K., Jimenez, F., Thomas, C. P., Smith, P., Hetz, R. A., Xue, H., Pati, S., Dash, P. K. & Cox, C. S., Jr. (2013). Autologous bone marrow mononuclear cells therapy attenuates activated microglial/macrophage response and improves spatial learning after traumatic brain injury. The journal of trauma and acute care surgery 75(3): 410-416.
28. Carlos, T. M., Clark, R. S., Franicola-Higgins, D., Schiding, J. K. & Kochanek, P. M. (1997). Expression of endothelial adhesion molecules and recruitment of neutrophils after traumatic brain injury in rats. Journal of leukocyte biology 61(3): 279-285.
29. Streit, W. J., Mrak, R. E. & Griffin, W. S. (2004). Microglia and neuroinflammation: a pathological perspective. Journal of neuroinflammation 1(1): 14.
30. Semmler, A., Hermann, S., Mormann, F., Weberpals, M., Paxian, S. A., Okulla, T., Schafers, M., Kummer, M. P., Klockgether, T. & Heneka, M. T. (2008). Sepsis causes neuroinflammation and concomitant decrease of cerebral metabolism. Journal of neuroinflammation 5: 38.
31. Wagner et al., Current Opinion in Biotechnology, 20:531-536, 2009.
32. U.S. Pat. No. 7,749,749
33. U.S. Pat. No. 8,852,923
34. U.S. Pat. No. 9,969,965

We claim:

1. An apparatus comprising:
a stationary member;
a magnetic field generator;
a controller; and
a cell culture medium located in the stationary member, wherein:
the cell culture medium comprises magnetic beads in a fluid; and
the controller is configured to control the magnetic generator to generate a variable magnetic field on the magnetic beads.

2. The apparatus of claim 1 further comprising a rotating member, wherein the rotating member is configured to rotate within the stationary member.

3. The apparatus of claim 2 wherein the cell culture medium is located between the stationary member and the rotating member.

4. The apparatus of claim 2 wherein:
the rotating member applies a rotational force to the magnetic beads via the fluid of the cell culture medium, wherein the rotational force is in a first direction; and.
the variable magnetic field applies a magnetic force to the magnetic beads, wherein the magnetic force is in a second direction that is different than the first direction.

5. The apparatus of claim 4 wherein the first direction is perpendicular to the second direction.

6. The apparatus of claim 4 wherein the magnetic force applied to the magnetic beads is greater than the rotational force applied to the magnetic beads.

7. The apparatus of claim 1 wherein the variable magnetic field is a pulsed magnetic field.

8. The apparatus of claim 2 wherein the rotating member comprises a plurality of discs.

9. The apparatus of claim 2 wherein the stationary member comprises a plurality of annular surfaces.

10. The apparatus of claim 9 wherein the plurality of annular surfaces are interdigitated with the plurality of discs.

11. The apparatus of claim 8 furthering comprising apertures extending through the plurality of discs.

12. The apparatus of claim 11 wherein the variable magnetic field moves the magnetic beads through the apertures extending through the plurality of discs.

13. The apparatus of claim 2 wherein the rotating member comprises a plurality of randomly oriented fibers.

14. The apparatus of claim 1 wherein the stationary member is configured as a toroidal container.

15. The apparatus of claim 14 wherein the magnetic field generator is configured as a series of coils wrapped around the toroidal container.

16. The apparatus of claim 15 wherein the controller is configured to pulse an electrical current through the series of coils wrapped around the toroidal container.

17. The apparatus of claim 16 wherein the magnetic beads are moved around the toroidal container via the electrical current pulsed through the series of coils.

18. The apparatus of claim 1 wherein the stationary member is configured as a linear tubular container.

19. The apparatus of claim 18 wherein the magnetic field generator is configured as a series of coils wrapped around the linear tubular container.

20. The apparatus of claim 19 wherein the controller is configured to pulse an electrical current through the series of coils wrapped around the toroidal container.

21. The apparatus of claim 20 wherein the magnetic beads are moved within the linear tubular container via the electrical current pulsed through the series of coils.

22. A method of culturing cells, the method comprising:
obtaining a cell culture medium comprising magnetic beads in a fluid; and
applying a variable magnetic force to the magnetic beads.

23. The method of claim 22 wherein the variable magnetic force is a pulsed magnetic force.

24. The method of claim 22 further comprising applying a rotational force to the magnetic beads via the fluid of the cell culture medium.

25. The method of claim 24 wherein:
the rotational force is applied to the magnetic beads in a first direction; and
the variable magnetic force is applied to the magnetic beads in a second direction that is different than the first direction.

26. The method of claim 25 wherein the first direction is perpendicular to the second direction.

27. The method of claim 25 wherein the magnetic force applied to the magnetic beads is greater than the rotational force applied to the magnetic beads.

28. The method of claim 24 wherein the rotational force is applied by a rotating member comprising a plurality of discs.

29. The method of claim 28 wherein the cell culture medium is contained in a stationary member comprising a plurality of annular surfaces.

30. The method of claim 29 wherein the plurality of annular surfaces are interdigitated with the plurality of discs.

31. The method of claim 30 wherein:
the plurality of discs comprises apertures extending through the plurality of discs; and
the variable magnetic field moves the magnetic beads through the apertures extending through the plurality of discs.

32. The method of claim 22 further comprising rotating a rotating member comprising a plurality of randomly oriented fibers to apply a rotational force to the magnetic beads via the fluid of the cell culture medium.

33. The method of claim 22 wherein the cell culture medium comprising magnetic beads in the fluid is contained in a toroidal container.

34. The method of claim 33 wherein the variable magnetic force is applied to the magnetic beads via a magnetic field generator configured as a series of coils wrapped around the toroidal container.

35. The method of claim 34 further comprising pulsing an electrical current through the series of coils wrapped around the toroidal container.

36. The method of claim 35 further comprising moving the magnetic beads around the toroidal container via the electrical current pulsed through the series of coils.

37. The method of claim 22 wherein the cell culture medium comprising magnetic beads in the fluid is contained in a linear tubular container.

38. The method of claim 37 wherein the variable magnetic force is applied to the magnetic beads via a magnetic field generator configured as a series of coils wrapped around the linear tubular container.

39. The method of claim 38 further comprising pulsing an electrical current through the series of coils wrapped around the linear tubular container.

40. The method of claim 39 further comprising moving the magnetic beads within the linear tubular container via the electrical current pulsed through the series of coils.
